SEQUENCE LISTING (2 sequences)

SEQ ID NO: 1 - protein, 121 aa, Synthetic construct
QVQLVQSGAEVKKPGSSVKVSCKASGYTFTSYWINWVRQAPGQGLEWIGAIYPGNGDTSYNQKFKDKATLTADKSSSTAYMQLSSLTSEDSAVYFCARGRGGWGTYAMDYWGQGTSVTVSS

SEQ ID NO: 2 - protein, 113 aa, Synthetic construct
DIVMTQSPDSLAVSLGERATINCKSSQSVLYSSNNKNYLAWYQQKPGKAPKLLIYDASTRHTGVPDRFSGSGSGTDFTLTISSLEPEDFAVYYCQQYNSYPLTFGAGTKLELK

(19) United States
(12) Patent Application Publication
(10) Pub. No.: US 2025/0388656 A1
(43) Pub. Date: Dec. 25, 2025

(54) SINGLE CHAIN VARIABLE FRAGMENT TRANSFERRIN FUSION PROTEIN (RNAT01) TO TREAT NEURODEGENERATIVE DISORDERS

(71) Applicants: Matthias Magoola, Kampala (UG); Sarfaraz K. Niazi, Deerfield, IL (US)

(72) Inventors: Matthias Magoola, Kampala (UG); Sarfaraz K. Niazi, Deerfield, IL (US)

(21) Appl. No.: 18/751,224

(22) Filed: Jun. 22, 2024

(51) Int. Cl. C07K 16/18; C07K 14/79

(52) U.S. Cl. CPC C07K 16/18; C07K 14/79; C07K 2317/622; C07K 2319/00

(57) ABSTRACT

Antibodies can treat neurodegenerative disorders (NDs) caused by misfolded proteins. Still, the blood-brain-barrier (BBB) resists their entry, a barrier that can be reduced by scFv instead of whole antibody and conjugating it with transferrin protein to induce transcytosis. While the scFv binds variable heavy and light chains with a non-cleavable linker, a cleavable linker between the scFv and transferrin protein can reduce exocytosis, enhancing the activity of the scFv.

### SEQUENCE LISTING

The instant application contains a Sequence Listing, which has been submitted electronically in ASCII format and is hereby incorporated by reference in its entirety. Said ASCII text file, created on Jun. 17, 2024, is named RNAT01_SequenceListing, and is 3 kb in size.

### BACKGROUND OF THE INVENTION

Many antibodies have been developed to treat central nervous system (CNS) disorders. However, no US Food and Drug Administration-approved monoclonal antibodies show efficacy in the brain following systemic administration. A primary reason is likely because the biological activity of therapeutic monoclonal antibodies and other recombinant protein therapeutics is limited by their ability to cross the blood-brain barrier (“BBB”), a term used to describe the unique properties of the microvasculature of the brain. Brain blood vessels are continuous, non-fenestrated vessels. They also contain additional properties that allow them to tightly regulate the movement of molecules, ions, and cells between the blood and the brain. This heavily restricting barrier capacity allows BBB endothelial cells to tightly regulate CNS homeostasis, which is critical for proper neuronal function and protecting the CNS from toxins, pathogens, inflammation, injury, and disease. However, the restrictive nature of the BBB also provides an obstacle to drug delivery to the CNS, especially for large proteins such as antibodies. Typically, less than 0.1% of the injected dose of IgG reaches the brain after peripheral administration, and it is difficult to achieve sufficient concentrations of antibodies in the brain to produce a therapeutic response. Developing antibodies that can penetrate the brain at pharmacologically relevant levels is a critical therapeutic goal for treating many CNS disorders.

Various methods have been investigated to improve brain exposure to various biologics, including antibodies, and avoid direct injection into the cerebrospinal fluid, which requires patient hospitalization, is highly invasive, and can easily cause infection. The most direct approach is to disrupt the tight junctions to allow paracellular passage. However, this is an unselective procedure for entering large molecules as it will enable entrance for the therapeutic agent and other blood components that could harm the brain environment. A similar approach is to use mannitol, an osmotic agent that causes shrinkage of the brain endothelial cells. However, this agent induces rapid, widespread BBB opening and exposes the brain tissue to potentially toxic circulatory system components. Another similar approach is the use of focused ultrasound. This is a method of transiently increasing the permeability of the BBB at specific brain regions. Still, again, it allows non-selective crossing of the BBB and, therefore, exposure to potentially harmful bloodborne substances.

Macromolecules such as monoclonal antibodies (“mAbs”) can utilize three types of vesicles to traverse brain endothelial cells: (i) clathrin-coated vesicles, (ii) caveolae domains generated from lipid rafts, and (iii) macropinocytotic vesicle. For transcytosis of large molecules, which primarily uses clathrin-coated vesicles, a receptor is required for uptake and trafficking across the brain endothelial cells.

This process has been termed “receptor-mediated transcytosis” (“RMT”). This is the only known vesicular system selective in cargo delivery, as it is coordinated by a specific receptor upon binding. This inherent selectivity is highly advantageous, and therefore, most attempts to transport large molecules across the BBB have used RMT-based approaches, for example, by using transferrin-containing nanoparticles or bispecific antibodies comprised of an arm against the receptor and a second against the therapeutic target.

Bispecific antibodies using RMT have only resulted in a relatively modest increase in the amount of mAb reaching the brain parenchyma, in most cases to the level of about 1% of the injected amount. Moreover, monovalent antibodies may reduce efficacy against the therapeutic target. Additionally, bispecific antibodies add significantly to the complexity of development and the cost of manufacture.

The mode of BBB receptor engagement can profoundly affect avidity, affinity, and valency of the antibody in the transport efficiency across the brain endothelial cells. Endogenous ligand binding also influence sthe receptor differently than an engineered mAb, which may bind to a different epitope.

When studying macromolecule transcytosis, the BBB has been regarded as a “black box,” with little attention to understanding the intracellular trafficking across brain endothelial cells. Thus, the regulation of intracellular transport through the transcytosis pathway in brain endothelial cells remains largely unexplored.

Many NDs are associated with the accumulation of misfolded or rogue proteins in the brain, such as Amyloid-beta and tau protein, Alpha-synuclein, Huntingtin, TDP-43, SOD1, FUS, and Prion protein, among others, leading to Alzheimer's disease (AD), Parkinson's disease (PD), amyotrophic lateral sclerosis (ALS), and Huntington's disease (HD). For some of these diseases, antibodies have been developed that are capable of binding or inactivating these proteins. Table 1 lists key identified proteins and antibodies to treat the NDs. However, there are dozens of other drugs under development to treat the NDs at various stages of the investigation, such as fusion constructs that can depend on protections (e.g., angiocept), affibodies (e.g., ABY025, SOBI002, ABY-035), affilins (e.g., PRS-010, PRS), U.S. Pat. No. 2,398,852 (Dezamizumab), AAB-001, (Bapineuzumab), ABBV-066 (Risankizumab), ABBV-323, ABBV-323, ABBV-8E12 (C2N 8E12), ABL-001 (Asciminib), ABL301, ABP-710 (Infliximab), ABT-165, ABT-414 (Depatuxizumab), ACZ885 (Canakinumab), ADC-1013, ADCT-402, ALD403 (Eptinezumab), ALX0061 (Vobarilizumab), AMG301 (Aimovig), AMG330, ANB020, ARGX-110, ARGX-111, ASKP1240 (Bleselumab), ATN103 (Ozoralizumab), BAN0805 (ABBV-0805), BAN2401 (mab158), BAY 1351 (Nerelimomab), B-E8 (Elsilimomab), Bertilimumab, BI 655064, BI 655066, BI 695500, BI695501, BIM 037 (Aducanumab), BIIB033 (Opicinumab), BIIB054 (Cinpanemab), BIIB074 (Vixotrigine), BIIB076, BMS-936561, Brolucizumab, BT-061 (Tregalizumab), C225 (cetuximab), CAT-192 (Metelimumab), CC-90002 (INBRX-103), CLNH11 (Pritumumab), cmt 412 (Priliximab), DB00028 (Gamunex), DB00073 (Rituximab), DB00095 (Efalizumab), DB00108 (Natalizumab), DB05459 (Briakinumab), DB05656 (Veltuzumab), DB06162 (Lumiliximab), DB06241 (Clenoliximab), DB06606 (Teplizumab), DB06650 (Ofatumumab), DB08935 (Obinutuzumab), DB09052 (Blinatumomab), DB11767 (Sarilumab), DB11803 (Sirukumab), DB11988 (Ocrelizumab), DB12053 (Visilizumab), DB12169 (Tralokinumab), DB12294 (Anrukinzumab), DB12636 (Samalizumab), DB12849 (Clazakizumab), DB13127 (Olokizumab), DB14004 (Tildrakizumab), DB14039 (Erenumab), DB14041 (Fremanezumab), DI-Leu16-IL2), Fazinumab, GC-1008 (Fresolimumab), GSE64382 (Itolizumab), GSK2862277, GSK3050002, GSK3174998, GSK3772847, GZ402668, HLX01, HLX03 (Adalimumab), Herceptin, Humax®-TAC-ADC, Humax-TAC-PBD, Inebilizumab, Inolimomab, IPH33, IPH52, JNJ-63709178, JNJ-63733657, JNJ-6400 57, KHK4083, KHK6 LY2062430 (Solanezumab), LY2599666, LY2951742 (Galcanezumab), LY3303560, MCLA-117, MEDI1341, MEDI1814, MEDI2070 (Brazikumab), MEN112, Mogamulizumab, NEOD001, Nesvacumab, Odulimomab, PF-04360365 (Ponezumab), PMN310, PRX002/R07046015, QAX576, REGN1979, REGN3500, RG1450 (Gantenerumab), RG6026, RG6100, RG6168, RG7412 (Crenzumab), RG7716, RG7828, RG7876, RG7935, RN624 (Tanezumab), RO 7105705, Rontalizumab, RYI 008 (Gerilimzumab), SAR228810, SAR3419 (Coltuximab ravtansine), SAR650984 (Isatuximab), SGN-CD19B, SGN-CD70A, Sym004 (Modotuximab), TAB-107 (Cedelizumab), TAB262 (Teneliximab), TAB-H16 (Dapirolizumab pegol), TAK573, TG-110 (Ublituximab), UCB4144, VX15/2503 (Pepinemab), xmab5574 (CD19), Xmab5871, Xoma 052, and (Gevokizumab), and Zanolimumab 0.050), anticalins (e.g., PRS-080, PRS-060, PRS-110, PRS343), atrimers (e.g., ATX 3105), avimers (e.g., AMG220), DARPins (e.g., MP0112, MP0260, MP0250, MP0274), fynomers (e.g., COVA322, COVA208, Knottin, Kuntiztdomain), bicyclic peptides, and other non-antibody proteins.

TABLE 1
Key rogue proteins in NDs and their antibodies
specific to the variations in the proteins

| Disease | Rogue Protein(s) | Antibody | Variations in Protein |
|---|---|---|---|
| Alzheimer's Disease | Amyloid-beta | Aducanumab | Aggregated forms of amyloid- |
| | | | beta (plaques) |
| Alzheimer's Disease | Amyloid-beta | Lecanemab | Soluble amyloid-beta |
| | | | protofibrils |
| Alzheimer's Disease | Amyloid-beta | Donanemab | Modified form of amyloid-beta |
| | | | with pyroglutamate at the N- |
| | | | terminus (N3pG) |
| Alzheimer's Disease | Amyloid-beta | Gantenerumab | Both fibrillar and non-fibrillar |
| | | | amyloid-beta |
| Alzheimer's Disease | Amyloid-beta | Solanezumab | Soluble amyloid-beta |
| | | | monomers |
| Alzheimer's Disease | Amyloid-beta | Crenezumab | Multiple forms of amyloid- |
| | | | beta, including oligomers and |
| | | | fibrils |
| Alzheimer's Disease | Tau | Semorinemab | Extracellular tau tangles |
| Alzheimer's Disease | Tau | Zagotenemab | Extracellular tau tangles |
| Alzheimer's Disease | Tau | Gosuranemab | N-terminal tau fragments |
| Alzheimer's Disease | Tau | Tilavonemab | N-terminal tau fragments |
| Parkinson's Disease | Alpha-synuclein | Prasinezumab | Aggregated alpha-synuclein |
| | | | (Lewy bodies) |
| Parkinson's Disease | Alpha-synuclein | Cinpanemab | Aggregated alpha-synuclein |
| | | | (Lewy bodies) |
| Parkinson's Disease | Alpha-synuclein | MEDI1341 | Monomeric and aggregated |
| | | | forms of alpha-synuclein |
| Parkinson's Disease | Alpha-synuclein | Exidavnemab | Toxic forms of alpha-synuclein |
| | | ABBV-0805 | |

Recently, monoclonal antibodies have come to the forefront as treatments of NDs based on their ability to remove rogue proteins selectively; however, after decades of efforts and billions invested, only two antibodies (Lecanemab and Aducanumab) have been approved, while dozens are under development. Aducanumab was recently withdrawn. Other antibodies, such as Bapineuzumab, Solanezumab, Crenezumab, and Gantenerumab, continue to struggle to demonstrate their efficacy.

The failure of antibodies to treat NDs is related to their low bioavailability in the brain, often cited to be around 0.01% of circulating serum concentrations of full antibodies and 0.1 to 0.4% for antibody fragments. This low bioavailability results from the robust BBB that protects the brain from exogenous and even most endogenous molecules. Consequently, the maximal brain concentration for peripherally dosed antibodies will always be insufficient to achieve the target engagement required for a therapeutic response.

Traditional methods to lower the BBB barrier by intracranial, intrathecal, or intraventricular injections and chemical disruption are neither practical nor reliable. However, reducing the size of antibodies from 150 kDA to around 10 kDA can be effective. Examples using the VH and VL portions of antibodies that bind, shorter forms of antibodies include scFv-CH (single-chain variable fragment), Single-chain variable fragment (scFv), scFV-CH3 (Minibodies), Diabody, sdAb (Single Domain Antibody), F(ab) 2 Fragments, F (ab) Fragment, Reduced IgG (rIgG). However, if the size of antibodies is reduced significantly, their disposition half-life in circulation decreases since they are filtered out of kidneys fast, partly making them ineffective. For this reason, the optimal size is those of scFv that have an additional advantage in that they do not induce microglia Fcγ receptor (FcγR)-mediated proinflammatory response and tissue damage in the central nervous system (CNS) because they lack the Fc portion of the immunoglobulin molecule.

The most plausible solution to improve the bioavailability of antibodies across the BBB is the exploitation of the transcytosis process, including receptor-mediated transcytosis (RMT), adsorptive-mediated transcytosis, and cell-mediated transcytosis. One of the most exploited receptors for RMT is the transferrin receptor (TfR), which naturally facilitates iron transport into the brain. Antibodies designed to target the TfR can hitch a ride across the BBB through this mechanism. Another example is the insulin receptor, which has also been targeted for RMT to deliver antibodies into the brain.

Antibodies can be engineered to bind to TfR directly or through transferrin. When engineering the antibody, it should preferably bind to a different epitope than Tf on the TfR to avoid interference with the endogenous process of iron delivery to the brain. Many TfR-antibodies bind the apical domain of TfR. It should have a moderate affinity for TfR in the nanomolar range. High affinity might result in a lower ability of the antibody to dissociate from TfR, which leads to sorting into lysosomes for degradation. The dissociation constant might be more critical than the association. Too low affinity might lead to poor ability of the antibody to bind TfR at the BBB and, consequently, poor brain delivery. Affinity can be pH-dependent, so the antibody binds TfR well at physiological pH but dissociates at lowered pH in the early endosome.

Conjugating with transferrin offers the best option of all the choices available for RMT, further optimized when using smaller-size antibodies that can be more readily transported across the BBB. While lacking the effector functions mediated by the Fc region, such as antibody-dependent cellular cytotoxicity (ADCC) and complement activation, the targeted binding of smaller antibodies allows better tissue penetration and, thus, overall clinical efficacy.

Erroneously, some assume that the antibodies conjugated with transferrin may reduce iron binding to transferrin and thus interfere in the transcytosis of iron in the brain. The transferrin concentration in human blood typically ranges from 200 to 360 mg/dL (2 to 3.6 g/L). Even though the binding of the antibody with transferrin will not interfere with iron binding to transferrin in conjugation, there is plenty of transferrin in the blood to compete well with the antibody-transferrin conjugate. This misperception has kept transferrin as the first choice of inducing transcytosis out of research.

Some smaller antibodies, like the single-chain variable fragments (scFvs), have the additional advantage of expression in bacteria, such as Escherichia coli, due to the simplicity, cost-effectiveness, and high yield of bacterial expression systems. Additionally, scFvs can be encoded easily as mRNA and delivered directly into cells, removing post-translational modification limitations when expressing using bacteria ensuring the correct modifications needed for stability and function. In eukaryotic cells, protein folding mechanisms and chaperones are in place to ensure proper folding of complex proteins. This increases the likelihood that a scFv will fold correctly and maintain its functional conformation. Eukaryotic cells, particularly the endoplasmic reticulum, provide an environment conducive to forming disulfide bonds, which are critical for the structural integrity of many antibodies. Eukaryotic cells have mechanisms to assist in the proper folding and solubility of proteins, reducing the risk of aggregation often seen in bacterial systems. When the scFv is produced directly inside the target cells, this approach bypasses the need for purification and refolding processes associated with bacterial expression systems, potentially leading to a more active and functional protein.

The scFv can also be delivered through encoding mRNA, particularly when combined with lipid nanoparticles (LNPs).

### Transferrin Binding

Binding transferrin to a single-chain variable fragment (scFv) involves deciding whether to attach it to the variable heavy (VH) or variable light (VL) chain. The decision depends on several factors, including the intended use, the structural implications, and the functional requirements. Here are some considerations to help us decide. Attaching transferrin to either the VH or VL chain could potentially affect the folding and stability of the scFv. The linker between VH and VL is designed to allow proper folding and antigen binding. Adding transferrin to one of these regions should not disrupt this structure.

When engineering an scFv, the C-terminus is a common site for attaching additional functional domains, such as transferrin, tags, or other proteins, because it is typically more accessible and does not interfere with the antigen-binding region: N-terminus—scFv (VH-VL)—Linker—Transferrin—C-terminus. The scFv (comprising VH and VL regions linked by a peptide linker) retains its antigen-binding capability in this configuration. A flexible linker connects the C-terminus of the scFv to the transferrin, ensuring both domains fold correctly and function independently.

Binding transferrin to an scFv with a cleavable linker that can be activated in the brain is a preferred approach to achieve targeted delivery and controlled release of the therapeutic agent. This strategy could potentially enhance the efficacy and specificity of the treatment. Examples of enzyme-sensitive linkers include matrix metalloproteinase (MMP)-sensitive linkers, cathepsin-sensitive linkers, and other brain-specific proteases. The pH-sensitive linkers that respond to the acidic microenvironment of specific brain regions or pathological conditions (e.g., tumors) can be used. An example sequence is GPLGVRG (cleaved by MMP-2 and MMP-9) Cathepsin-Sensitive Linkers; cathepsin is a protease found in lysosomes and is often upregulated in cancer. An example sequence is GGFGL (cleaved by Cathepsin B). The pH-sensitive Linkers are designed to be cleaved or undergo conformational changes in response to acidic environments, such as those found in tumors or inflamed tissues. Aspartate-proline linkers can undergo cleavage at acidic pH.

Another example is DPG (dipalmitoylglycerol), a redox-sensitive linker that contain disulfide bonds that can be reduced in high glutathione concentrations, often found in cells' cytoplasm. Disulfide Bond Linkers example is CPC (Cysteine-Proline-Cysteine). These linkers can be connected as follows:
N-terminus—scFv (VH-VL)—Flexible Linker—Cleavage Linker—Flexible Linker—Transferrin

The primary function of the scFv is to bind to its target antigen. The attachment of transferrin should not interfere with the antigen-binding site. Attaching transferrin to the C-terminus of the VH or VL chain might be advantageous, away from the antigen-binding region. Transferrin should remain accessible for its function, including binding to transferrin receptors. Attaching transferrin to the VH chain's C-terminus might provide better accessibility than the VL chain, depending on the overall design and orientation of the scFv.

Using multiple types of cleavage linkers that respond to different environmental triggers can increase the specificity and efficiency of cleavage, especially in complex environments like the brain or tumor microenvironments. This strategy can provide a more controlled and targeted release of therapeutic agents by ensuring that the cleavage occurs only in the presence of the desired conditions.

An example of a single cleavage linker at the C-terminus might be sufficient for certain applications: N-terminus—Transferrin—(Flexible Linker)—(MMP-Sensitive Linker)—scFv—VH—(Flexible Linker)—scFv—VL—C-terminus

An example configuration of a fusion protein with multiple cleavage linkers: N-terminus—scFv VH—(Flexible Linker)-scFv VL—(Flexible Linker)—MMP-Sensitive Linker)—(Flexible Linker)—(pH-Sensitive Linker)—(Flexible Linker)—(Redox-Sensitive Linker)—(Flexible Linker)—Transferrin—C-terminus

The ideal flexible linker is (G4S)3. The MMP-sensitive linker is GPLGVRG; the pH-sensitive linker is DPG; the Redox-sensitive linker is CPC.

The pH in different brain compartments is crucial for designing effective mRNA constructs with pH-sensitive linkers. The pH of the extracellular fluid (ECF) in the brain is typically around 7.4, which is similar to the physiological pH of the blood. Brain cells' intracellular fluid (ICF) is slightly more acidic, generally maintaining a pH of around 7.0-7.2. The environment is significantly more acidic within cellular compartments such as endosomes and lysosomes. Early endosomes have a pH of approximately 6.0-6.5, while late endosomes and lysosomes exhibit even lower pH values, ranging from 4.5 to 5.0. These varying pH levels are essential considerations for mRNA design, particularly for incorporating pH-sensitive linkers. For example, a polyhistidine (His) linker, which responds to slightly acidic pH, is suitable for targeting endosomal and lysosomal environments within brain cells.

The Asp-Pro-Gly (DPG) linker is another example, as it is cleavable under mildly acidic conditions, making it ideal for use in the brain's intracellular compartments. These linkers ensure controlled cleavage and release of therapeutic agents, optimizing the effectiveness of mRNA constructs in brain applications.

Matrix metalloproteinases (MMPs) are critical brain enzymes involved in tissue remodeling, neuronal network formation, and maintaining blood-brain barrier integrity. These zinc-dependent endopeptidases can degrade extracellular matrix components and are tightly regulated by tissue inhibitors of metalloproteinases (TIMPs). Among the various MMPs, MMP-9 and MMP-2 are particularly significant in the central nervous system, as they are implicated in neuroinflammation, blood-brain barrier permeability, and synaptic plasticity.

Redox-sensitive linkers, like those responsive to MMP activity, are valuable in mRNA design for brain applications because they target the specific enzymatic environment. An example of such a linker is a peptide sequence that is cleavable by MMP-9. This linker can be integrated into mRNA constructs to enable controlled release and activation of therapeutic agents in response to MMP activity, which is often elevated in various neurological conditions, including neuroinflammation and brain injuries.

Specific examples of MMP-sensitive linkers that can be used in mRNA design for applications in the brain, responding to the presence of matrix metalloproteinases: Pro-Leu-Gly-Leu-Trp-Ala: Cleaved by MMP-2 and MMP-9; Gly-Pro-Leu-Gly-Ile-Ala-Gly-Gln: Cleaved by MMP-9; Arg-Pro-Leu-Ala Cleaved by MMP-2 and MMP-9.

Redox-sensitive linkers are essential in designing mRNA constructs for brain applications because they respond to the reductive environment within cells. One widely used redox-sensitive linker is the disulfide bond (Cys-Cys), which can be cleaved by reducing agents such as glutathione. Glutathione is abundant in the intracellular environment, especially within the cytosol of brain cells. It is a tripeptide composed of glutamine, cysteine, and glycine, playing a crucial role in maintaining the redox balance and protecting neurons from oxidative stress. The high glutathione concentration in the brain ensures that disulfide bonds in mRNA constructs can be efficiently cleaved, facilitating the controlled release of therapeutic agents or functional domains. This targeted cleavage mechanism enhances the specificity and efficacy of mRNA-based therapies, making disulfide bonds an ideal choice for redox-sensitive linkers in brain applications.

Cathepsin-sensitive linkers are peptide sequences designed to be cleaved by specific cathepsins, facilitating the controlled release of therapeutic agents in environments where these enzymes are active. Examples include Gly-Phe-Leu-Gly Ala-Leu-Ala-Leu.

For optimal function, the binding with transferrin should not alter the binding of scFv with target protein such as amyloid beta (Abeta); to test this hypothesis, a well-known and approved antibody, aducanumab was docked with or without transferrin conjugate. The results of protein-protein complex analysis reveal significant differences in binding affinity and interaction characteristics (Table 1) upon conjugation with transferrin. The ΔG value was calculated at −18.1 kcal mol−1, with a corresponding Kd of 5.10E⁻¹⁴ M at ° C. for the Aducanumab-Abeta complex. The interface analysis demonstrated varying interactions, with notable contributions from charged-charged and apolar-apolar interactions. We observed enhanced binding affinity and altered interaction profiles upon conjugation with transferrin using different linkers. Specifically, the Transferrin-(G4S) 3-Aducanumab-Abeta complex exhibited the highest ΔG value (−29.2 kcal mol⁻¹) and the lowest Kd (4.10E⁻²² M at ° C.), indicative of stronger binding. Interface analysis further revealed increased interactions across all categories, particularly in polar-apolar and apolar-apolar interactions. These findings underscore the importance of linker length in modulating protein binding and highlight the potential of our approach for optimizing drug-protein interactions. These findings underscore the critical role of linker length in modulating binding properties. Comparative analysis using (G4S) or (G4S)3 linker has revealed superior flexibility with the latter, facilitating unimpeded binding to proteins with minimal steric hindrance around the amyloid-binding pocket. It can be further attached to MMP, DPG, and CPC linkers. Furthermore, these findings indicate that linking the antibody's light chain with transferrin does not compromise antibody binding to brain proteins nor impede amyloid's accessibility to its binding domain. (Table 2)

TABLE 2
The binding properties of transferrin conjugates of drugs developed for treating NDs.

| | | | | | | | | | | |
|---|---|---|---|---|---|---|---|---|---|---|
| Protein- | ΔG | | ICs | ICs | ICs | ICs | ICs | ICs | | |
| protein | (kcal | Kd (M) | charged- | charged- | charged- | polar- | polar- | apolar- | NIS | NIS |
| complex | mol−1) | at ° C. | charged | polar | apolar | polar | apolar | apolar | charged | apolar |

Aducanumab

| | | | | | | | | | | |
|---|---|---|---|---|---|---|---|---|---|---|
| Aducanumab- | −18.1 | 5.10e⁻¹⁴ | 22 | 20 | 64 | 1 | 19 | 29 | 21.20 | 40.10 |
| Aβ | | | | | | | | | | |
| Transferrin- | −21.3 | 2.60e⁻¹⁶ | 39 | 27 | 83 | 1 | 18 | 43 | 28.50 | 35.50 |
| (G4S)- | | | | | | | | | | |
| Aducanumab- | | | | | | | | | | |
| Aβ | | | | | | | | | | |
| Transferrin- | −29.2 | 4.10e⁻²² | 32 | 33 | 136 | 7 | 38 | 79 | 28.90 | 35.70 |
| (G4S)₃- | | | | | | | | | | |
| Aducanumab- | | | | | | | | | | |
| Aβ | | | | | | | | | | |

Donanemab

| | | | | | | | | | | |
|---|---|---|---|---|---|---|---|---|---|---|
| Donanemab- | −13.4 | 1.40e⁻¹⁰ | 8 | 7 | 30 | 2 | 18 | 33 | 21.39 | 37.50 |
| Aβ | | | | | | | | | | |
| Transferrin- | −15.5 | 4.20e⁻¹² | 8 | 3 | 43 | 1 | 23 | 57 | 28.81 | 35.00 |
| (G4S)- | | | | | | | | | | |
| Donanemab- | | | | | | | | | | |
| Aβ | | | | | | | | | | |
| Transferrin- | −30.8 | 2.40e⁻²³ | 36 | 12 | 104 | 8 | 58 | 155 | 28.59 | 35.10 |
| (G4S)₃- | | | | | | | | | | |
| Donanemab- | | | | | | | | | | |
| Aβ | | | | | | | | | | |

Crenezumab

| | | | | | | | | | | |
|---|---|---|---|---|---|---|---|---|---|---|
| Crenezumab- | −13.1 | 2.6e⁻¹⁰ | 12 | 18 | 48 | 4 | 10 | 24 | 20.46 | 40.06 |
| Aβ | | | | | | | | | | |
| Transferrin- | −20.3 | 1.3e⁻¹⁵ | 27 | 36 | 89 | 5 | 20 | 35 | 27.53 | 36.66 |
| (G4S)- | | | | | | | | | | |
| Crenezumab- | | | | | | | | | | |
| Aβ | | | | | | | | | | |
| Transferrin- | −28.4 | 1.6e⁻²¹ | 33 | 54 | 97 | 20 | 63 | 95 | 27.87 | 37.12 |
| (G4S)₃- | | | | | | | | | | |
| Crenezumab- | | | | | | | | | | |
| Aβ | | | | | | | | | | |

Gantenerumab

| | | | | | | | | | | |
|---|---|---|---|---|---|---|---|---|---|---|
| Gantenerumab- | −13.1 | 2.6e⁻¹⁰ | 12 | 18 | 48 | 4 | 10 | 24 | 20.46 | 40.06 |
| Aβ | | | | | | | | | | |
| Transferrin- | −25.6 | 1.6e⁻¹⁹ | 43 | 31 | 99 | 5 | 32 | 51 | 28.26 | 35.75 |
| (G4S)- | | | | | | | | | | |
| Gantenerumab- | | | | | | | | | | |
| Aβ | | | | | | | | | | |
| Transferrin- | −19.1 | 9.7e⁻¹⁵ | 26 | 29 | 74 | 4 | 21 | 35 | 27.99 | 36.48 |
| (G4S)₃- | | | | | | | | | | |
| Gantenerumab- | | | | | | | | | | |
| Aβ | | | | | | | | | | |

Solanezumab

| | | | | | | | | | | |
|---|---|---|---|---|---|---|---|---|---|---|
| Solanezumab- | −25.6 | 1.6e⁻¹⁹ | 34 | 20 | 101 | 6 | 36 | 58 | 19.60 | 42.33 |
| Aβ | | | | | | | | | | |
| Transferrin- | −23.6 | 4.8e⁻¹⁸ | 15 | 35 | 53 | 23 | 71 | 68 | 28.14 | 36.17 |
| (G4S)- | | | | | | | | | | |
| Solanezumab- | | | | | | | | | | |
| Aβ | | | | | | | | | | |
| Transferrin- | −27.8 | 4e⁻²¹ | 39 | 25 | 105 | 12 | 47 | 40 | 27.97 | 36.25 |
| (G4S)₃- | | | | | | | | | | |
| Solanezumab- | | | | | | | | | | |
| Aβ | | | | | | | | | | |

ΔG (kcal mol−1) is the predicted value of the binding affinity, expressed in kilocalories per mole, indicating the energy change associated with forming a complex between molecules. It represents the difference in energy between the bound and unbound states of a ligand and its target, indicating the spontaneity of the binding process. A more negative ΔG signifies stronger binding affinity, meaning the interaction is thermodynamically favorable, and the ligand binds tightly and specifically to the target. Conversely, a less negative or positive ΔG suggests weaker binding affinity and less favorable interaction, indicating the ligand may dissociate more readily. A negative ΔG also indicates spontaneous binding, essential for effective ligand-target interactions in biological systems. At the same time, a positive ΔG denotes non-spontaneous binding, which typically requires energy input and is undesirable in drug design. Additionally, a more negative ΔG implies a more stable ligand-target complex, crucial for drug efficacy as it ensures prolonged interaction with the target. In contrast, a less negative or positive ΔG suggests an unstable complex prone to dissociation. Free energy calculations predict the likelihood of successful binding, aiding drug discovery and development by allowing researchers to prioritize compounds with favorable binding properties. By understanding free energy changes, researchers can optimize ligands to enhance binding affinity and stability, leading to more effective drugs. Free energy also comprises enthalpic and entropic contributions, helping to dissect the driving forces behind binding, such as hydrogen bonds and van der Waals interactions for enthalpy and the release of water molecules for entropy. It is the most critical indicator of the strength of binding.

Kd (M or molarity indicating the molar concentration of the ligand) at the default 25° C., representing the equilibrium concentration of dissociated molecules in solution. Mathematically, it is given by the ratio of the rate constants for dissociation (koff) and association (kon): Kd=koff/kon. A higher Kd value indicates that a higher ligand concentration is required to occupy half of the binding sites. This suggests a weaker binding affinity between the ligand and the target. In practical terms, the ligand dissociates more readily from the target, implying faster dissociation. Thus, a ligand with a lower Kd is generally preferred, indicating a more robust and stable interaction with the target.

Intermolecular Contacts (ICs) are the specific points of interaction between the ligand and the target molecule within a defined distance threshold (5.5 Å in this case). Generally, it indicates a more robust and extensive interaction between the ligand and the target. Lower number of ICs: This may indicate weaker binding affinity and less stable interactions. More contacts can mean a more stable and specific binding. Charged-Charged Interactions between oppositely charged groups (ionic interactions) and Charged-Polar Interactions between charged and polar (partially charged) groups are typically more favorable as they involve strong ionic and hydrogen bonding interactions, respectively—charged-Apolar Interactions between charged groups and nonpolar groups (less common, often less favorable).

Polar-polar interactions occur between hydrophilic groups that can form hydrogen bonds and dipole-dipole interactions. These interactions are significant because hydrogen bonds are strong and directional, contributing to the stability and specificity of the ligand-protein complex. Dipole-dipole interactions enhance binding affinity by aligning dipoles in a favorable orientation. Additionally, polar-polar interactions improve solubility in aqueous environments, which is crucial for biological activity and drug delivery, and help form hydration shells around the binding site, influencing binding dynamics and stability. Higher scores in polar-polar interactions generally indicate stronger, more specific binding due to increased hydrogen bonding and dipole interactions. Lower scores suggest weaker interactions and potentially less stable binding.

Polar-apolar interactions occur between hydrophilic and hydrophobic groups. These interactions help position the ligand correctly within the binding site, potentially causing conformational changes that facilitate stronger interactions. While weaker than other interactions, they are transitional in the initial binding stages. Polar-apolar interactions also influence the ligand's solubility and bioavailability by balancing polar regions that enhance solubility with apolar regions that improve membrane permeability. Overall, they contribute to the binding kinetics by balancing the forces involved in the binding process. Higher scores in polar-apolar interactions indicate better positioning and initial ligand recognition. In comparison, lower scores suggest less effective interactions that may not optimally guide the ligand into the binding site.

Polar-polar interactions occur between hydrophilic groups that can form hydrogen bonds and dipole-dipole interactions. These interactions are significant because hydrogen bonds are strong and directional, contributing to the stability and specificity of the ligand-protein complex. Dipole-dipole interactions enhance binding affinity by aligning dipoles in a favorable orientation. Additionally, polar-polar interactions improve solubility in aqueous environments, which is crucial for biological activity and drug delivery, and help form hydration shells around the binding site, influencing binding dynamics and stability. Higher scores in polar-polar interactions generally indicate stronger, more specific binding due to increased hydrogen bonding and dipole interactions. Lower scores suggest weaker interactions and potentially less stable binding.

Polar-apolar interactions occur between hydrophilic and hydrophobic groups. These interactions help position the ligand correctly within the binding site, potentially causing conformational changes that facilitate stronger interactions. While weaker than other interactions, they play a transitional role in the initial binding stages. Polar-apolar interactions also influence the ligand's solubility and bioavailability by balancing polar regions that enhance solubility with apolar regions that improve membrane permeability. Overall, they contribute to the binding kinetics by balancing the forces involved in the binding process. Higher scores in polar-apolar interactions indicate better positioning and initial ligand recognition. In comparison, lower scores suggest less effective interactions that may not optimally guide the ligand into the binding site.

A higher number of ICs is generally better, indicating more robust and stable interactions. Favorable contacts (charged-charged, polar-polar, and apolar-apolar) are significant.

Non-interacting surface (NIS %) refers to the proportion of the ligand or target surface that does not participate in intermolecular interactions. NIS Charged: The percentage of the charged surface area not involved in interactions. It Indicates that a more significant portion of the charged surface is not involved in binding, which might suggest suboptimal electrostatic complementarity. Lower NIS Charged is better, indicating that the charged regions effectively participate in interactions, contributing to binding affinity. Higher NIS Apolar demonstrates that a more significant portion of the apolar surface is not involved in binding. This might suggest that the hydrophobic interactions are not maximized. Lower NIS Apolar indicates better utilization of apolar regions for binding, which is favorable for hydrophobic interactions. It is better, as it demonstrates that the apolar regions effectively participate in hydrophobic interactions, contributing to binding stability.

In a binding study, a higher number of intermolecular contacts (ICs), particularly those involving favorable interactions (charged-charged, polar-polar, apolar-apolar), is generally desirable. Lower NIS charged and NIS apolar values are preferred, as they indicate better binding utilization of the molecular surfaces. This combination correlates with more robust, stable, and specific ligand-target interactions.

### RNAT01.

RNAT01 is the claimed scFv with Sequence No. 1, which is a VH chain, and Sequence No. 2, which is a VL chain, when combined, are capable of binding specifically to various forms of cerebral proteins such as amyloid beta, tau, and alpha-synuclein proteins in multiple forms causing a variety of NDs.

TABLE 1
Sequence of VH and VL for scFv

| SEQ # | SEQUENCE |
|---|---|
| SEQUENCE | QVQLVQSGAEVKKPGSSVKVSCKASGYTFTSYWINWVR |
| NO. 1 VH | QAPGQGLEWIGAIYPGNGDTSYNQKFKDKATLTADKSS |
| | STAYMQLSSLTSEDSAVYFCARGRGGWGTYAMDYWGQG |
| | TSVTVSS |
| SEQUENCE | DIVMTQSPDSLAVSLGERATINCKSSQSVLYSSNNKNY |
| NO. 2 VL | LAWYQQKPGKAPKLLIYDASTRHTGVPDRFSGSGSGTD |
| | FTLTISSLEPEDFAVYYCQQYNSYPLTFGAGTKLELK |

Designing and generating a single-chain fragment variable (scFv) fusion protein (RNAT01) antibody requires choosing an appropriate linker, preferably a non-cleavable linker to join the VH and VL and a cleavable linker to connect the scFv with transferrin protein.

1. An antigen-binding single chain variable fragment (scFv) transferrin fusion protein (RNAT01) comprising a variable heavy chain (SEQUENCE NO. 1) and a variable light chain (SEQUENCE NO: 2), linked by a flexible non-cleavable linker to form an scFv that is further conjugated with transferrin protein (P02787 TRFE_HUMAN) preferably using a cleavable linker, to form a scFv fusion protein.

2. An antigen-binding single chain variable fragment (scFv) transferrin fusion (RNAT01) of claim 1, wherein the transferrin protein is fused to either the carboxyl-terminus or the amino-terminus of light or heavy chain of the antibody either monovalently or multivalently.

3. The antigen-binding single chain variable fragment (scFv) transferrin fusion protein (RNAT01) of claim 1, wherein the binding of the scFv with transferrin protein enhances the binding efficiency of RNAT01 with amyloid-beta, tau, and alpha-synuclein proteins, preferably monomeric and aggregated forms of alpha-synuclein.

4. The antigen-binding single chain variable fragment (scFv) transferrin fusion protein (RNAT01) of claim 1, wherein the flexible non-cleavable linker comprises an amino acid, polyethylene glycol, or a polymer, preferably glycine-glycine-glycine-glycine-serine (G4S), or its multiples thereof.

5. The antigen-binding single chain variable fragment (scFv) transferrin fusion protein (RNAT01) of claim 1, wherein the flexible linker cleavable linker comprises a hydrazone, disulfide, or peptide, preferably Gly-Gly-Phe-Gly (GGFG), or its multiples thereof.

6. The antigen-binding single chain variable fragment (scFv) transferrin fusion protein (RNAT01) of claim 1, wherein the said RNAT01 can bind amyloid-beta, tau, and alpha-synuclein proteins, preferably monomeric and aggregated forms of alpha-synuclein.

7. The antigen-binding single chain variable fragment (scFv) transferrin fusion protein (RNAT01) of claim 1, wherein the said RNAT01 can induce transcytosis through transferrin receptor binding into the brain.

8. The antigen-binding single chain variable fragment (scFv) transferrin fusion protein (RNAT01) of claim 1, wherein the said RNAT01 is produced using recombinant expression in bacteria or mammalian cells.

9. The antigen-binding single chain variable fragment (scFv) transferrin fusion protein (RNAT01) of claim 1, wherein RNAT01 is encoded by an mRNA composition and delivered through lipid nanoparticles.
